(19) United States
(12) Patent Application Publication
(10) Pub. No.: US 2022/0361033 A1
(43) Pub. Date: Nov. 10, 2022

(54) METHODS AND SYSTEMS FOR TRANSMITTING DATA BETWEEN DEVICES WORN BY SWIMMERS

(71) Applicant: Matsya LLC, La Jolla, CA (US)

(72) Inventors: Abhinav SRIDHAR, La Jolla, CA (US); John R. ZEBRYK, JR., Southbridge, MA (US); Mark STEPHENS, Marietta, GA (US)

(21) Appl. No.: 17/735,166

(22) Filed: May 3, 2022

Related U.S. Application Data
(60) Provisional application No. 63/184,567, filed on May 5, 2021.

(51) Int. Cl. H04W 28/02; H04B 1/3827; H04R 1/10; A61B 5/00

(52) U.S. Cl. CPC H04W 28/0231; H04B 1/385; H04R 1/1041; A61B 5/0002; H04B 2001/3861; H04R 2420/07

(57) ABSTRACT

Methods and systems for improving and/or enabling wireless communication and data transfer between electronic devices worn by swimmers and both wireless networks and wireless headphones or other worn devices. The methods and systems disclosed detect when conditions are sufficient for wireless communication depending on the position of a worn electronic device relative to the surface of the water and/or wireless headphones and enables burst data transfer between devices and/or between a device and a wireless network during such times.

### CROSS REFERENCE TO RELATED APPLICATIONS

This application is related to U.S. Provisional Patent Application No. 63/184,567 filed on May 5, 2021, titled “Method for Providing a Reliable Near Real-Time Radio Connection Between a Wrist Worn Device and a Head Worn Device for Swimmers” which is incorporated herein by reference.

### FIELD

The disclosed technology relates generally to electronic communications and more particularly to systems and methods for reliably transmitting data wirelessly to swimmers.

### BACKGROUND

Swimmers, especially those performing multiple laps for exercise, have long desired to listen to music to relieve monotony and to provide inspiration. Competitive swimmers are passionate about receiving real-time performance metrics and feedback. Although “smartwatches” or other portable electronic devices are ubiquitous for both groups, currently there is no practical way to effectively utilize many of the capabilities of these powerful devices due to the significant attenuation of radio signals underwater.

Currently, the most common way of listening to music or of receiving other digital information while swimming is through use of an electronic device (smartphone, smart watch, or a dedicated audio player) which is strapped to the head or other part of the body and either directly or wirelessly connected to headphones or earbuds. Such systems suffer from many disadvantages. Music must either be uploaded wirelessly or manually transferred prior to swimming for some systems. During actual swimming, changing volume, track number, play list, etc., is very awkward as the controls are located at the top of the head and not visible. Such systems also require the device to be worn on the head and/or have a wire connecting the player device to the headphones which can be uncomfortable and awkward.

Wireless headphones and devices such as smartphones or smartwatches capable of wireless communication typically function poorly in aquatic environments. Most wireless radio frequencies commonly used by such devices (such as the 2.4 GHz band used by Bluetooth® protocol) typically propagate poorly in water. (Bluetooth® is a registered trademark of the non-profit Bluetooth Special Interest Group located in Kirkland, Wash., USA). Wireless protocols which operate in the 2.4-5.0 GHz range such as Bluetooth® and Wi-Fi might have a range measured in meters when operating in air but a range measured in centimeters when the radio waves must travel through water. Such limitations inhibit the functionality of wireless headphones if the source such as a smart watch is worn anywhere on the body where it does not remain in constant, close proximity to the headphones. Wearing a source such as a smartwatch on the wrist or arm will repeatedly take the source outside the effective communication range with the headphones as the swimmer's arm moves through a typical swimming stroke.

The issue of signal attenuation through water also makes most other wireless communication functionality of devices such as smartphones and smart watches unreliable. Applications or services which normally rely on the ability to stream data wirelessly either through Wi-Fi or a broadband cellular network may function poorly or not at all on a device which is repeatedly submerged in water such as when a swimmer's arm moves through a typical swimming stroke. Workarounds for such limitations such as preloading music files onto a device and using a wired connection between headphones and a player device might be suitable in some circumstances, but applications or services which require constant, near-constant, or frequent connection to remote services such as music streaming services or applications which monitor telemetric data for the purpose of providing feedback to a swimmer require other solutions.

Some solutions such as signal repeaters either worn by a swimmer or placed around a pool can be useful in some circumstances. Such systems only work when the swimmer is near a repeater which means reception might be intermittent or depend on the swimmer maintaining a steady pace so they are never out of range of a repeater for longer than expected by the system. In other circumstances such as free swimming in a lake or other large body of water there is no pool edge or shore on which to place a repeater. There remains a need for systems and methods for improving the wireless communication capabilities for electronic devices worn while swimming.

### SUMMARY

In one aspect, systems and methods for transmitting data wirelessly to a swimmer wearing an electronic device where the worn device is moved alternatingly between areas of high signal strength (typically above the water) and low signal strength (typically below the water). The worn device continuously detects if the device is in an area of high signal strength or low signal strength. When in areas of high signal strength the device receives and buffers data from a wireless data source at a first data rate. When in areas of low signal strength and/or high signal strength the device transmits data to a receiving device at a second data rate, where the first data rate is higher than the second data rate. The wearable electronic device may be a smart watch and the receiving device may be wireless headphones. In other aspects the wearable device and the receiving device are a single device. In still other aspects, a second type of data is received, buffered, and transmitted in addition to the first type of data, where one type of data may be selectively prioritized over the other type of data as desired. In further aspects, data prioritization may change depending on predetermined conditions being met.

In another aspect, systems for streaming data wirelessly to and from a swimmer in an aquatic environment involving a wearable electronic device having a memory for storing data and at least one antenna for connecting to one or more data networks and to one or more receiving devices. The wearable electronic device being capable of constantly detecting if the device is in an area of high signal strength (typically above water) or low signal strength (typically below water) from a predetermined wireless network and selectively receiving and buffering data in the memory at a first data rate when the wearable electronic device is in an area of high signal strength, and transmitting data from the wearable electronic device at a second data rate to a receiving device, the receiving device being capable of receiving data from the wearable electronic device, and where the first data rate is higher than the second data rate. In other aspects the wearable electronic device is a smart watch and the receiving device is wireless headphones. In still other aspects the wearable electronic device and the receiving device are components of a single device. In further aspects the wearable electronic device is capable of selectively receiving and buffering a second type of data in the memory when the wearable electronic device is in an area of high signal strength and of transmitting the second type of data to a receiving device. The first type of data may be prioritized over the second type of data consistently or selectively until certain predetermined conditions are satisfied such as a period of time passing or certain biometric or telemetry conditions for the swimmer have been satisfied.

### BRIEF DESCRIPTION OF THE DRAWINGS

FIG. 1 is an illustration of a swimmer's arm positions during a swimming stroke.

FIG. 2 is an illustration of the phases of a typical swimming stroke.

FIG. 3a is a top diagrammatic view of a swimmer's head and hand positions.

FIG. 3b is a side diagrammatic view of head and hand positions of FIG. 3a.

FIG. 4 is an illustration of a swimmer and wireless network sources.

FIG. 5 is a block diagram of a receiving device usable with an example of the disclosed invention.

FIG. 6 is a block diagram of a smart watch device usable with an example of the disclosed invention.

FIG. 7 is a block diagram of a data transfer system according to one example of the disclosed invention.

FIG. 8 shows a graph of signal strength variability during swim strokes.

FIG. 9a is a circuit diagram of a wearable device according to one example of the disclosed invention.

FIG. 9b is a continuation of the circuit diagram of FIG. 9a.

FIG. 9c is a continuation of the circuit diagram of FIG. 9b.

### DESCRIPTION

For the purposes of promoting an understanding of the principles of the claimed technology and presenting its currently understood best mode of operation, reference will now be made to the embodiments illustrated in the drawings and specific language will be used to describe the same. It will nevertheless be understood that no limitation of the scope of the claimed technology is thereby intended, with such alterations and further modifications in the illustrated device and such further applications of the principles of the claimed technology as illustrated therein being contemplated as would normally occur to one skilled in the art to which the claimed technology relates.

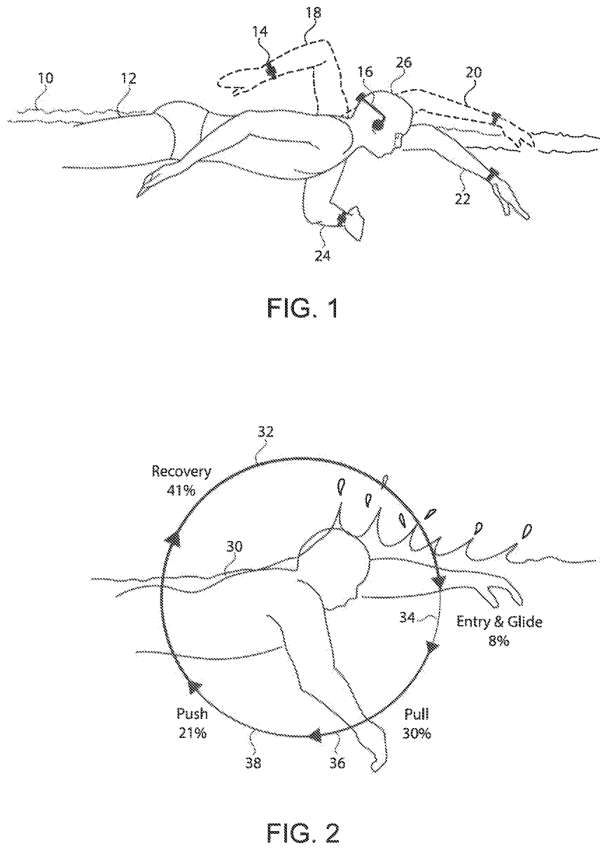

As seen in FIG. 1 the motions of swimmer 12 performing a typical swimming stroke involve a swimmer's arm 18, 20, 22, 24 passing through a range of positions both relative to the swimmer's head 26 and relative to the surface of the water 10. In this particular example, arm positions typical for the stroke known as the front crawl are shown (also commonly known as the Australian crawl or freestyle). Other swimming strokes such as the butterfly, breaststroke, backstroke, and the like also have cycles where a swimmer's arm is above/below the surface of the water and/or near/far from the swimmer's head. The systems and methods disclosed herein would also work with other swimming strokes and the use of the front crawl is for illustrative purposes only.

As shown in FIG. 1, during a typical swimming stroke a swimmer's arm will be in a plurality of different positions relative to the swimmer's head and relative to the surface of the water. Relative to the surface of the water, some arm positions (18, 20) will place the arm above the water 10 where radio waves can easily be received or sent, some arm positions (22) relatively close to the surface of the water 10 where radio waves might be able to still reach a wrist-worn device such as a smart watch 14, and still other arm positions (24) where the wrist worn device 14 is too far below the water's surface 10 to reliably receive useful signals. Relative to the swimmer's head and to headphones 16 or ear bud devices, the arm at some positions (18, 24) will be close enough that wireless communications between the wrist mounted device 14 and headphones 16 will be possible whereas in other positions (22) the wrist mounted device may be too far from the headphones 16 for reliable communications. In still other positions (20) communications may be possible if both the wrist mounted device 14 and headphones are above the water 10 but not possible if one or both have passed below the surface of the water 10.

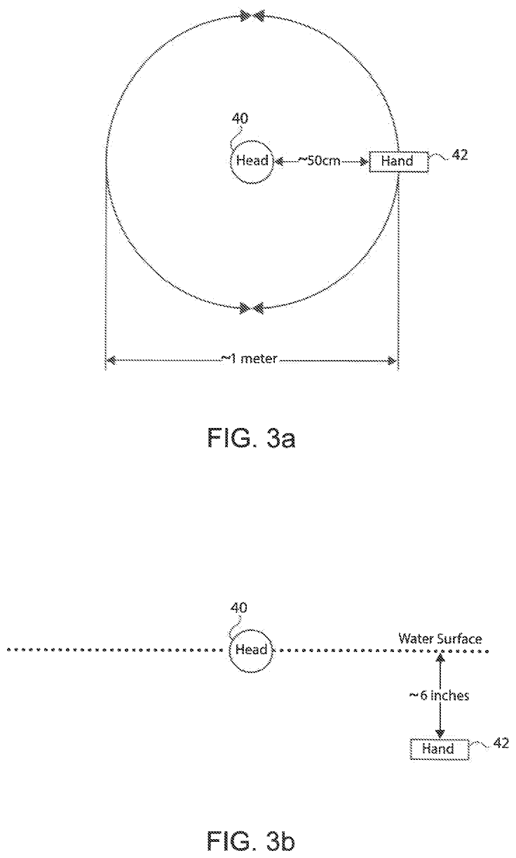

A typical swimming stroke consists of a repeated series of a single cycle where the swimmer's hand begins at a starting position (e.g., position 20 in FIG. 1), moves through a series of intermediary positions (e.g., positions 22, 24, and 18 in FIG. 1) before returning to the starting position. Such strokes typically include periodic intervals coincidental with breathing wherein the hand is either above the water or slightly below (typically 0 to 6 inches). Generally, independent of stroke rate, this period of opportunity for error free communications is approximately 50% of the total stroke duration depending on the stroke being performed. For a swimmer 30 performing the front crawl at 60 strokes per minute, for example, this window of opportunity is approximately 30 seconds long and includes most of the “recovery” 32 and “entry and glide” 34 phases as shown in FIG. 2.

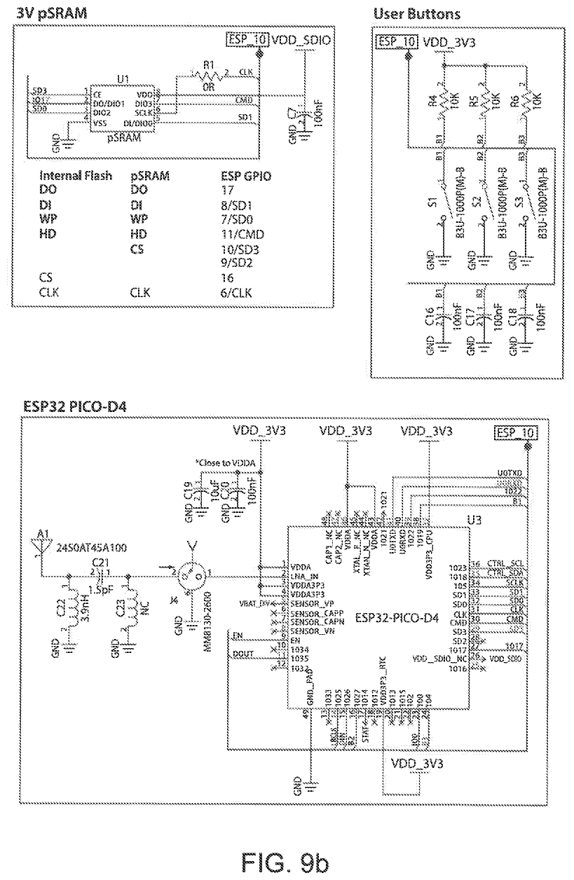

Underwater, radio waves are attenuated and absorbed by many orders of magnitude as compared to in air. As this attenuation increases logarithmically with distance, underwater communications are not practical for many applications. However, it is observed, in swimming, the depth that the wrist is submerged is relatively shallow except during the propulsion phase of the stroke (the “push” 38 and “pull” 36 phases as shown in FIG. 2). During the initial portions of the “recovery” 32 and “entry and glide” 34 phases, the radio pathway is typically sufficient to support successful communications. This is an additional opportunity for those periods wherein the wrist is not totally airborne. An example of how signal strength varies during a series of typical swimming strokes is shown in FIG. 8. In this particular example, a received signal strength threshold 110 is necessary for successful transmission of data to worn device. Signal strength below the threshold 112 is too weak as too much if the signal is attenuated by the worn device being below the water at a particular portion of the stroke. Signal strength above the threshold 114 is sufficient for a successful transmission of data when the worn device is above the water or sufficiently shallow that signal attenuation is sufficiently small. The exact duration of each duration of sufficiently strong signal strength and the period between such times of sufficiently strong signal strength will vary according to user, stroke being used, length of time swimming, and the like. For a typical swimmer performing the front crawl the duration of each period of strong connectivity might be from 0.4 to 2.5 seconds with periods of poor connectivity in between from 0.5 to 4.0 seconds. For other strokes and/or other swimmers these periods may be longer or shorter. When swimmers perform actions other than normal swimming strokes such as diving into the water or performing a flip turn and glide after reaching the end of a pool periods of poor or no connectivity may be even longer, upwards of 20 seconds or more.

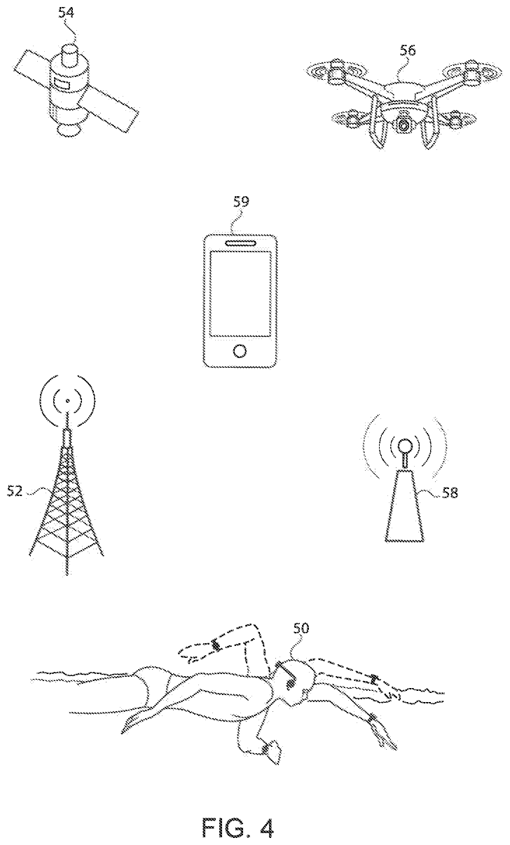

As shown in FIG. 3a, the radio mounted on the head 40 has an operating range significantly greater than one meter for successful air to air communications. However, when the wrist radio 42 is submerged this operating range decreases significantly with depth. Even so, when shown from the side (FIG. 3b), radio waves do penetrate the surface sufficiently to support successful communications. This penetration depth varies with frequency, but for 2.4 GHz Bluetooth® radio signals this depth is approximately six inches within a one-meter radius about the head. Such an arrangement may be less useful in some situations (such as when a swimmer is performing the backstroke) and more useful in other situations (such as when a swimmer is performing the breaststroke where the hands may never fully be above the water).

In one example of the disclosed methods and systems data buffering is utilized in conjunction with burst transmission of data. During the propulsion portion of a stroke, when radio communications is not possible, data to be sent is temporarily stored in memory. When communication does become possible during a stroke cycle the stored data is transmitted in a burst and optionally deleted from memory. This cycle of buffering during periods of poor/no communication and burst transmission during periods where communication is possible then repeats as the swim stroke cycle is repeated. The quality of the radio communication link between the source device (such as a smart watch, smart phone, etc.) and the receiving device (such as headphones) is continuously monitored so as to take advantage of periods of strong communication links when they occur. Continuous monitoring by the system in this particular example allows for a particular swimmer to vary or change the stroke being used without interrupting the presentation of data as the system is always looking for periods where communication is possible and taking advantage of them when they occur.

In some examples, a buffering algorithm is specifically designed to work reliably in aquatic environments where disconnections and long periods of poor/no connectivity are common. When such a system is tasked with transmitting music data the audio chunk (i.e., a group of bits associated with music data) size and the number of audio chunks to fetch from the remote server are set dynamically based on the signal conditions. The estimated wi-fi Radio Signal Strength Indicator (RSSI) or signal strength is used to either trigger aggressive buffering (e.g., until the free heap memory or RAM is full) or to wait and poll the wireless interface for the previous access point to come back online. Traditional systems typically attempt to connect to other networks if there is a disconnection from the current network source. Buffering logic is implemented in the background as playback continues uninterrupted. Traditional buffering systems cannot handle source network (Wi-Fi or Bluetooth®) disconnects while seamlessly playing back content. Any network interruptions will cause the playback to pause. They also do not implement the opportunistic buffering mechanism based on Radio Frequency (RF) signal strength and other network-related factors such as fading, interference, multipath, and the like. Previous systems are usually timer based or based on buffer health. Also, the amount of data buffered in previous systems is small (typically a few seconds), whereas the some of the systems disclosed herein may maximize the worn device's memory utilization to support long durations where the device can be offline/out of network contact for minutes without depleting the device's buffer of data.

The present systems and methods also allow for uses which require different volumes and types of data and/or data from different types of sources (e.g., stored on a nearby electronic device or streamed over a network from a remote web site). The amount of data transferred may vary according to the information being transferred. For example, if the data being transferred is musical in content the data rate of a burst transmission would typically be higher than what is required for normal playback so as to prevent the receiving device (e.g., headphones) from running out of data and causing a pause or skip in the music. Additionally, such data may be handled differently if the source is a local device or if it is from a streaming-style music service which may limit how fast data may be provided by the service. In other applications, various types of data may be gathered using instruments also included on a worn device such as magnetometers, accelerometers, GPS devices, and the like for determining telemetric data about a user's swimming such as speed, distance traveled, and the like. Data collected by such instruments may also be used to calculate additional information about a user such as acceleration/deceleration, glide time, flutter frequency, porpoise efficiency, and the like. Other instruments such as may also be used to collect biometric data about a user such as heart rate, blood oxygenation level, respiration rate, and the like. In such examples, the transfer of swimming telemetry data (e.g., lap times, distance traveled, time remaining, and the like) and/or biometric data (heart rate, blood oxygenation levels, respiration rate, and the like) the data rate of a burst transmission may be lower as an interruption or pause in the communication of such data is less important and/or the information being transferred involves less data.

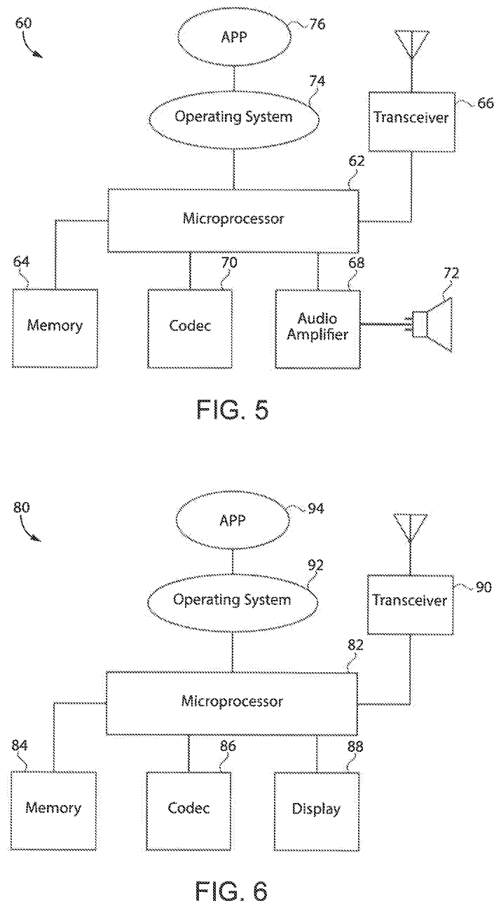

The same systems and methods discussed herein for use with transferring data from a worn device such as a smart watch or smart phone to a worn receiving device such as headphones may also be used to manage data transfer to/from a remote source such as a wireless network from/to the worn device 50. As shown in FIG. 4, such remote sources could include wireless phone networks 52, satellites 54, drones 56, Wi-Fi networks or other access points 58, mobile hot spots 59, and the like. In one example a system according to the present disclosure might prioritize transfer of data between the worn device such as a smart watch and a wireless network when the worn device is out of the water (when wireless signals are strongest) such as positions 18 and 20 in FIG. 1 but prioritize data transfer from the worn device to a receiving device such as headphones when the worn device is below the water (so as to block signals from a wireless network) but the worn device is sufficiently close to the receiving device so as to allow for communication. In other examples, the system might adjust the priority between data transfer between the worn device and a wireless network and data going out of the worn device to a worn receiving device such as headphones depending on a plurality of conditions such as signal strength, data buffer sizes (in the worn device and/or in the worn receiver), predetermined priority status assigned by the user (e.g., prioritize a music streaming service over swimming telemetry data). In other examples, the predetermined priority status of data may be dynamic in nature. That is, priority might be given to one type of data (e.g., music streaming) for a predetermined period of time (e.g., 10 minutes) and/or until a particular condition is satisfied (e.g., when 10 laps are completed, when the user's heart rate exceeds a predetermined threshold, or when the user's pace falls outside a predetermined range) and then a second type of data may be prioritized (e.g., telemetric or biometric data). In still other examples, data type priority may change depending on how much data of a particular type is stored in a buffer (e.g., music data is prioritized until there is 10 laps of biometric data stored then biometric data is prioritized).

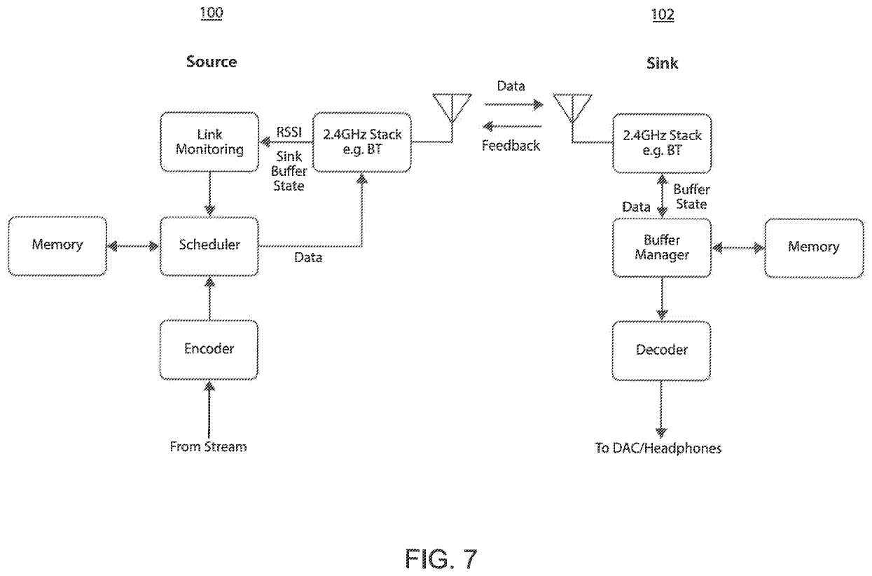

A schematic diagram of a typical worn receiving device such as auto headphones are shown in FIG. 5. In this particular example, the headphones 60 include a microprocessor 62 operationally connected to a memory 64, a codec 70 suitable for decoding music or other audio data to be played, an amplifier 68 which is operationally connected to one or more speaker units 72, and an antenna/transceiver 66 such as a Bluetooth® transceiver or a Wi-Fi transceiver. Loaded into the memory is an operating system 74 which runs on the microprocessor 62 and which is running one of the data control and management applications 76 disclosed herein.

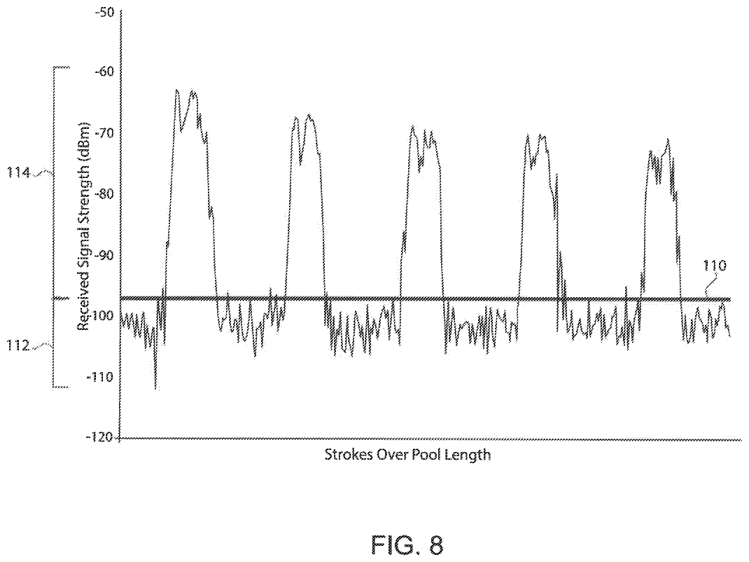

A schematic diagram of a typical worn device 80 such as a smart watch or smart phone is shown in FIG. 6. In this particular example, the worn device 80 includes a microprocessor operationally connected to a memory 84, a codec 86 suitable for encoding/decoding music or other audio data, an display 88 (optionally a touch display), and an antenna/transceiver 90 such as a Bluetooth® transceiver or a Wi-Fi transceiver. Loaded into the memory 84 is an operating system 92 which runs on the microprocessor 82 and which is running one of the data control and management applications 94 disclosed herein. The receiving device 60 and worn device 80 shown in FIGS. 5-6 are for illustrative purposes only. Devices having different configurations and/or components may also be used to practice the systems and methods disclosed herein.

In one example of the disclosed systems and methods, in performing an operation to listen to music a swimmer opens an application, selects Music Mode, and then the play list and/or the individual songs/tones/tracks desired. Upon entering the water, the swimmer then selects “Play”. In another example operation, a swimmer opens the application, selects Telemetry Mode, and then selects the stroke element(s) to be reported, type of subliminal feedback desired, etc. Upon entering the water and assuming the starting position, the swimmer selects “Start”. During swimming, if the swimmer had selected stroke analysis for example, the application would analyze readings from the smart watch's accelerometer and using natural language voice prompts provide short summary reports of the various portions of the stroke involved. These could include “efficiency 62%”, “kick rate too fast”, “pace is 60”, and the like.

In addition to voice annunciation, some examples of the disclosed systems also support apparent and subliminal suggestions of auditory patterns representing optimal strokes and/or specific segments of strokes. In one example, a tone of variable frequency and magnitude of the swimmer's personal ideal stroke rate. Matching the cadence of the idea, the swimmer would hear a pleasant sound pattern. However, if not matching the ideal rate, the sound pattern would be discordant and unpleasant. This real-time feedback would allow the swimmer to adjust their movements accordingly. A separate pattern could be used for kick rate which the swimmer would also try to match. Depending on the objectives of the swimmer (e.g., competitive vs exercise) all three types of audio could be intermingled. Music could be periodically over-written with selected swim metric reports, and subliminal audio patterns could continue in the background in one example.

In another example, the worn device smart phone or smart watch, utilizes a 2.4 GHz radio internal to the device and the Bluetooth® protocol for communications. In other examples, other frequencies and/or protocols may be used. For example, the 5 GHz band (5.030 to 5.990 GHz) is also frequently used for data transmission and communications and could also be used by the disclosed systems and methods. If the application is oriented towards audio annunciation, a A2DP Profile may be employed. For a file transfer-based approach an OBEX FTP protocol could be employed instead. Other data transfer protocols and/or handling methods may also be used in other examples of the disclosed systems and methods.

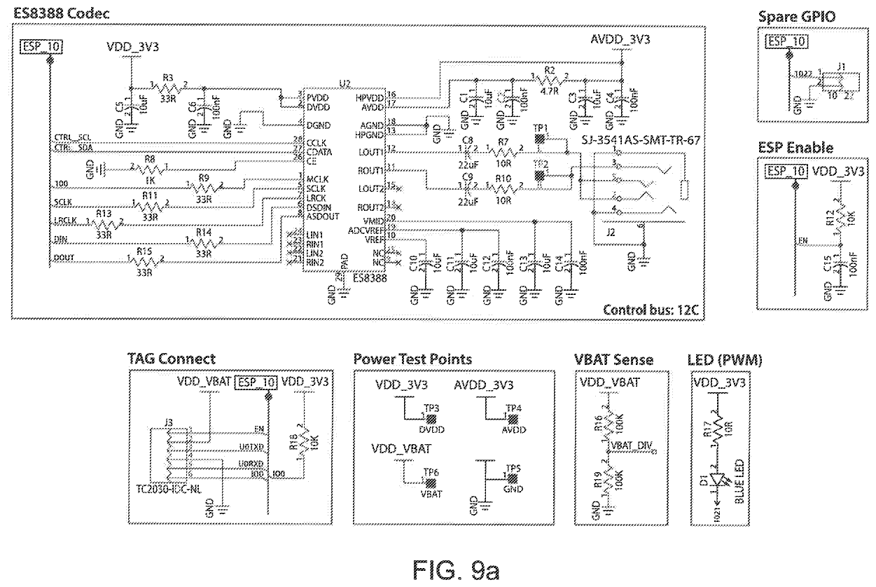

In order to implement an opportunistic communication method as disclosed herein, specialized application software may be used. As seen in FIG. 7, a source application 100 and a sink application 102 may be used where the source application 100 manages the transmission of data from the smart phone and keeps track of what data was received and acknowledged by the sink application 102. The sink application 102 manages the incoming data from the source application 100 and presents it to the audio subsystem for presentation to the swimmer. This can be either through conventional acoustic headphones or through bone conduction headphones.

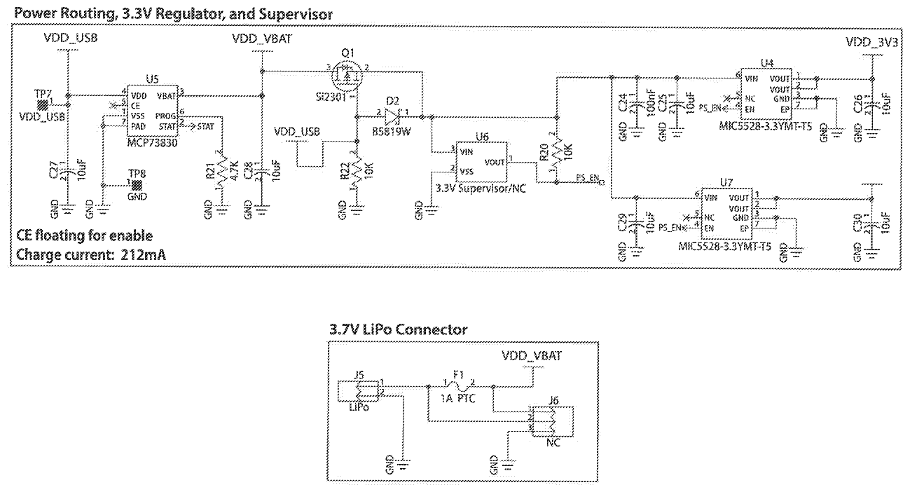

In addition to playing continuous uninterrupted music or other audio, the disclosed systems and methods also enable reporting of the swimmer's performance in virtual real-time whilst swimming. Typically waterproof smart watches or other devices incorporate an accelerometer, many measurements can be calculated using physics. Using either pre-recorded phrases, or the text to speech engines included with most smartphones and smart watches, one or more of following reports or analysis can be communicated to the swimmer: SWOLF score (number of strokes over one lap or averaged over “N” laps); efficiency score (calories/BTUs expended over one lap or averaged over “N” laps); average kick rate (flutter frequency); dive distance; flip time; glide time/deceleration (time to stroke); stroke power (acceleration/swimmer weight); and/or drag (deceleration during the return stroke). These reports can be communicated to the swimmer in real time or near real time. Optionally, such information may be calculated and stored to be relayed at preset time intervals (e.g., every 10 minutes), when predetermined conditions are met (e.g., every 100 meters traveled), or when certain events occur (e.g., upon a flip turn at the end of a pool). The circuit diagram 120 of one example of a portable electronic device suitable for use with the methods and systems disclosed herein is shown in FIG. 9, although other suitable devices may also be used.

While the claimed technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the claimed technology are desired to be protected.

What is claimed is:

1. A method of streaming data to a worn electronic device in an aquatic setting, comprising:
moving a wearable electronic device between areas of high signal strength and low signal strength;
detecting if the wearable electronic device is in an area of high signal strength or low signal strength;
receiving and buffering data in the wearable electronic device at a first data rate when the wearable electronic device is in an area of high signal strength; and
transmitting data from the wearable electronic device at a second data rate to a receiving device when the wearable electronic device is in an area of low signal strength or high signal strength;
wherein the first data rate is higher than the second data rate.

2. The method of claim 1, wherein the wearable electronic device is a smart watch.

3. The method of claim 1, wherein the receiving device is wireless headphones.

4. The method of claim 1, wherein the periods of high signal strength are when the wearable electronic device is located above water and the periods of low signal strength are when the wearable electronic device is located below water.

5. The method of claim 1, wherein the detecting step is continuous.

6. The method of claim 1, wherein a second type of data is received and transmitted in addition to the first type of data.

7. The method of claim 6, wherein the first type of data is prioritized over the second type of data during the receiving and transmitting.

8. The method of claim 7, wherein the first type of data is prioritized over the second type of data during the receiving and transmitting until a predetermined condition is satisfied and then the second type of data is prioritized over the first type of data.

9. The method of claim 1, wherein the data is selected from the group including musical stream data, biometric data relating to a swimmer wearing the wearable electronic device, and telemetry relating to a swimmer wearing the wearable electronic device.

10. The method of claim 7, wherein the first type of data is musical streaming data and second type of data is one or more of biometric data relating to a swimmer wearing the wearable electronic device and telemetry relating to a swimmer wearing the wearable electronic device.

11. A system for streaming data wirelessly to and from a swimmer in an aquatic environment, comprising:
a wearable electronic device having a memory for storing data and an antenna for connecting to one or more data networks and to one or more receiving devices, the wearable electronic device being capable of constantly detecting if the device is in an area of high signal strength or low signal strength from a predetermined wireless network and selectively receiving and buffering data in the memory at a first data rate when the wearable electronic device is in an area of high signal strength, and transmitting data from the wearable electronic device at a second data rate to a receiving device; and
a wearable receiving device capable of receiving data from the wearable electronic device;
wherein the first data rate is higher than the second data rate.

12. The system of claim 11, wherein the wearable electronic device is a smart watch and the receiving device is wireless headphones.

13. The system of claim 11, wherein the wearable electronic device and the receiving device are components of a single device.

14. The system of claim 11, wherein the wearable electronic device is capable of selectively receiving and buffering a second type of data in the memory when the wearable electronic device is in an area of high signal strength and of transmitting the second type of data to a receiving device.

15. The system of claim 14, wherein receiving and transmitting the first type of data is prioritized over the second type of data.

16. The system of claim 15, receiving and transmitting the first type of data is prioritized over the second type of data until a predetermined condition is satisfied and then the second type of data is prioritized over the first type of data.

17. The system of claim 16, wherein the predetermined condition is a period of time passing.

18. The system of claim 16, wherein the predetermined condition is one or more of a biometric telemetric condition relating to the swimmer is satisfied.
